(19) United States
(12) Patent Application Publication
(10) Pub. No.: US 2008/0255440 A1
(43) Pub. Date: Oct. 16, 2008

(54) Method of Forming a Sterilized Sensor Package and a Sterilized Sensor Package

(75) Inventors: Michael Eilersen, Copenhagen (DK); Thomas Buch-Rasmussen, Gentofte (DK); Kristian Glejbol, Glostrup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/660,035

(22) PCT Filed: Jul. 13, 2005
(86) PCT No.: PCT/EP2005/053357

§ 371 Date: Feb. 20, 2008

(30) Foreign Application Priority Data
Aug. 10, 2004 (DK) PA 2004 01212

(51) Int. Cl. A61B 5/00; B65B 55/08

(52) U.S. Cl. 600/373; 53/428

(57) ABSTRACT

A sensor package is provided comprising an implantable sensor having an electrode area and an electric contact area, the package comprising a shielding packaging enclosing at least the electrode area of the sensor while exposing the electric contact area. The sensor package fascilitates handling of the implantable sensor and provides for the possibility of sterilising the electric contact area leaving the electrode area protected.

This invention relates to a method of sterilising an electrochemical sensor comprising an electrode area and an electric contact area. The invention relates in particular to sterilisation of integrated sensor assemblies employing electrochemical sensors and electronic circuits, in particular integrated sensor assemblies employing transcutaneous electrochemical sensors suitable for in vivo measurement of metabolites.

Medical devices have changed considerably over the years. For instance, the devices are becoming more complicated and use many different materials, such that autoclaving, chemical sterilisation, or the like, is no longer possible, since the processes would likely destroy the materials of the medical device or fail to reach areas not readily accessible after construction of the medical device.

In recent years, a variety of electrochemical sensors have been developed for in vivo measurements of metabolites. Most prominent among these are glucose sensors developed for measurement of blood glucose (BG) levels in a diabetic patient. Below the invention will be exemplified using glucose sensors. The invention is, however, by no means limited to glucose sensors, but applies in general to transcutaneous electrochemical sensors.

BG information is of the utmost importance to diabetics, as BG information is instrumental in the adjustment of the treatment regimen.

The conventional way to obtain BG information is by applying minute amounts of blood to test strips, whereupon the BG value can be read using a meter. A new development is transcutaneous sensors, where the sensor is implanted under the skin. As the sensor is always in contact with biological fluids, this opens for the possibility of continuous measurements. Continuous BG measurements (CGM) obtained with little or no delay will be particularly useful in numerous ways. First of all, the continuous monitoring will help to prevent hypoglycaemic incidents and thus contribute to a vast increase in the quality of life for the diabetic patient.

Furthermore, BG readings can be used in conjunction with automated or semi-automated medication infusion pumps of the external type. This will allow the patient to have a near normal lifestyle, thus eliminating the problems usually associated with diabetes.

After deposition of the conducting material, the working electrode of the system is now covered with a biological material which ensures specificity of the sensor towards the analyte of interest. If the sensor is used for detection of glucose, the biological material most often applied is glucose oxidase (GOD).

If the passive sensor is fitted with active electronics, the sensor/electronics assembly is referred to as a sensor assembly. Sensor assemblies are well known in the art and often used to reduce the overall sensitivity of the system towards noise. A sensor assembly is eg described in US2003/0100821A1.

After completion of the sensor assembly, the sensor will have to be sterilised. Three different strategies presently exist for sterilisation, namely radiation sterilisation, steam sterilisation and sterilisation by exposure to ethylene oxide (EtO). If biological material (eg GOD) is used to ensure the specificity of the sensor system, radiation sterilisation is most often the only viable strategy as other sterilisation schemes will destroy the biological materials essential to the sensor function.

Conversely to the biological material, the electronic part of a sensor assembly is sensitive to the exposure of energetic radiation as radiation deteriorates the semi-conducting circuits which are the vital part of the electronics. Most notably the amplifiers amplifying the minute currents from the sensor will be sensible to deterioration during sterilisation.

The problem of sterilisation devices employing electronics as well as radiation-sensitive parts is addressed in U.S. Pat. No. 6,594,156, where radiation sterilisation is made possible by shielding of the radiation sensitive parts.

Radiation sterilisation is an expensive method per se, and the method disclosed in U.S. Pat. No. 6,594,156 involves that the electronic circuit is to be radiated from two opposed sides.

It is the object of the invention to provide a method that, on the one hand, facilitates sterilisation of a disposable assembly comprising sensor and electronics and, on the other, increases the handling reliability of disposable sensors to be coupled to multiple-use electronics by the users themselves.

This object is accomplished in that at least the electrode area is enclosed in a shielding packaging that is impermeable to micro-organisms, in such a manner that the electric contact area extends outside the shielding packaging; and that the part of the sensor which is situated outside the shielding packaging is sterilised.

By this method it is accomplished, in a production line, that the electrode area of the sensor and the electronics can be sterilised by each their preferred sterilisation process due to the shielding packaging enclosing the electrode area protecting the electrode, eg against a gas that can advantageously be used for sterilising the electronics.

The method according to the invention also enables a user who is to couple his multiple-use electronics to a new disposable sensor to handle the sensor without an ensuing risk of infecting the electrode area, the latter being protected by the shielding packaging while the user couples the multiple-use electronics to the new disposable senor, whose electric coupling area projects outside the shielding packaging. Following mounting of the electronics, the shielding packaging is removed and the sterile sensor area can be implanted in the body.

For some uses of a disposable sensor with integral electronics it is not a requirement that the electronics as such are sterile; rather it suffices that the electrode area is sterile. For other uses the electronics must also be sterile, and following interconnection of the electronics with the electrode areas of the sensor, a sterilisation of the electronics is performed, eg by means of ethylene oxide, the shielding packaging protecting the glucose oxidase, which is an example of a substance that may be present in the electrode area, against the ethylene oxide gas. Then the entire sensor with integral electronics can be enclosed in a further shielding packaging.

It is a well-known problem that the radiation from the radiation sterilisation influences the biological substances that are often used on the electrode area of the electrochemical sensors, such as glucose oxidase if an electrode for measuring the glucose concentration in the body fluid is concerned. According to the invention this problem is largely obviated in that, firstly, a series of sensors is produced that are sterilised as explained above, and then test samples are extracted and measurements performed, by which it is possible find representative calibration parameters for this entire series of sensors. Then all of the remaining sensors in the series can be provided with an information carrier containing the calibration parameters. For safety reasons it is important that the information carrier is present in the sensor as such to be coupled to the associated electronics to take into account the calibration parameters rather than the calibration parameters being written on eg the sensor packaging. The information carrier may be passive circuits, eg an antenna circuit, whose information contents can be read by means of a radio transmitter in the electronics coupled thereto or the information carrier can be active electric circuits that are connected to the circuits in the electronics that perform calculations.

The invention also relates to a sensor package that may either be without electronics, wherein a user is to connect the sensor to his multiple-use electronics himself, or it may be a sensor assembly; ie an electrochemical sensor with electronics coupled thereto.

Over time, electronics will become so inexpensive that it is financially viable to produce such disposable equipment, but for the time being the electronics are still so expensive that most users use disposable electrodes and multiple-use electronics. When the user himself is to couple the new sensor to the old electronics, there is a high risk of the sensor becoming infected, in particular in view of the fact that diabetics are often elderly people and/or visually impaired.

The object of the sensor package according to the invention is to provide an implantable sensor that can be used in connection with electronics and addressing both the problem associated with a user using multiple-use electronics and the issue of also accomplishing an inexpensive and efficient sterilisation of the electronic components that are used in combination with the sensor in the context of a disposable assembly—which undoubtedly will become very widely used in the years to come.

This object is accomplished in that base packaging is provided that shields at least the electrode areas of the sensor; and that the electric coupling areas are situated outside the base packaging.

Hereby it is firstly accomplished that a user using multiple-use electronics is able to handle the electrical coupling without an ensuing risk of the sensor becoming infected as the sensor is, during that procedure, still protected by said first base housing. In the context of manufacture of disposable assemblies including electronics, the sensor can be sterilised by means of radiation, while the electronics can be sterilised separately by means of some other and less expensive sterilisation procedure. Said packaging can be a film or a form-stable wall being at least impermeable to micro-organisms. An additional packaging may be provided that covers those parts of the sensor that project outside the base packaging. This may be expedient for long-term storage in store, and the additional packaging can either adjoin the base packaging or completely enclose the sensor including the base packaging.

Preferably the base packaging includes means for transdermal insertion of the electrodes of the sensor. Such means may be a needle or other means for implanting electrodes into the body, see eg Danish patent application No. PA 2003 017432.

According to one embodiment the base packaging and the additional packaging may adjoin each other along an endless rim comprising a tearing line or a rupture zone. Preferably the base packaging is constituted by a form stable housing, which has considerable significance to the user, in that it makes it easier for him/her to handle the sensor, and in that it is considerably easier, in a production line, to assemble a sterile sensor and an electronic circuit.

For instance, the user may seize the form-stable housing and introduce needle and sensor and withdraw the needle or other means for inserting the sensor. In particular in case the sensor and electronics are later to be assembled in a production line, it may be expedient that the base packaging constitutes an integral housing and that a rupture zone is provided some place between the electrode area and the electric coupling area of the sensor. The manufacture of the disposable sensor assembly will preferably comprise a pair of polymeric half-parts configured for being joined and intended for receiving a sensor and a needle for implanting the sensor. Such housing is preferably provided with a mechanical coupling means for cooperating with a housing containing an electronic circuit, where the electronic circuit can be sterilised by use of a method that is far less expensive than radiation sterilisation. According to the invention, it is possible to put electronic circuits and sterile sensors in storage and assemble them at a later stage in order to subsequently sterilise the circuits in a convenient manner that would normally destroy the sensor which is, however, in accordance with the invention protected by the shielding packaging, while the electric coupling area of the sensor projects outside the shielding packaging in such a manner as to enable it to be coupled to the electronic circuits.

A further embodiment of a sensor for being interconnected with multiple-use electronics may comprise a form-stable housing with a top part and a lower part, which parts can, by means of cooperating slide faces, be shifted at least a distance into each other corresponding to the expanse of the electric coupling area of the sensor, which projects through the slit in both the top and the lower part, whereby a barrier is accomplished which is double-impermeable to micro-organisms when the top and lower parts are interconnected. The top part can be configured such that it can be used both for inserting the needle and for withdrawing it clear of the electric coupling area of the sensor.

By a further embodiment which is suitable both for producing sensor assemblies and for being used by a user in combination with multiple-use electronics, the base packaging comprises a tube which is closed at the one end and the other end of which is configured for receiving a part of the sensor which is configured as a piston. The piston can slide in the tube and does not at least in its initial position allow micro-organisms to enter the tube. This embodiment accomplish a very simple sterile packaging, while simultaneously the use enables many other advantages—see below.

The tube can be closed by a tear-off label, and the piston may serve as support for a sensor either configured as a needle or in any other manner configured for inserting a flexible sensor. In this manner embodiments are enabled that are, from a production point of view, very similar to each other and wherein the one is suitable for further (subsequent) production-line mounting, whereas the other is suitable for being mounted by a user.

Said tube may in itself constitute a part of the sensor to be adhered to the skin of a user.

The invention will be explained in further detail by the following description of exemplary embodiments, reference being made to the drawing, wherein

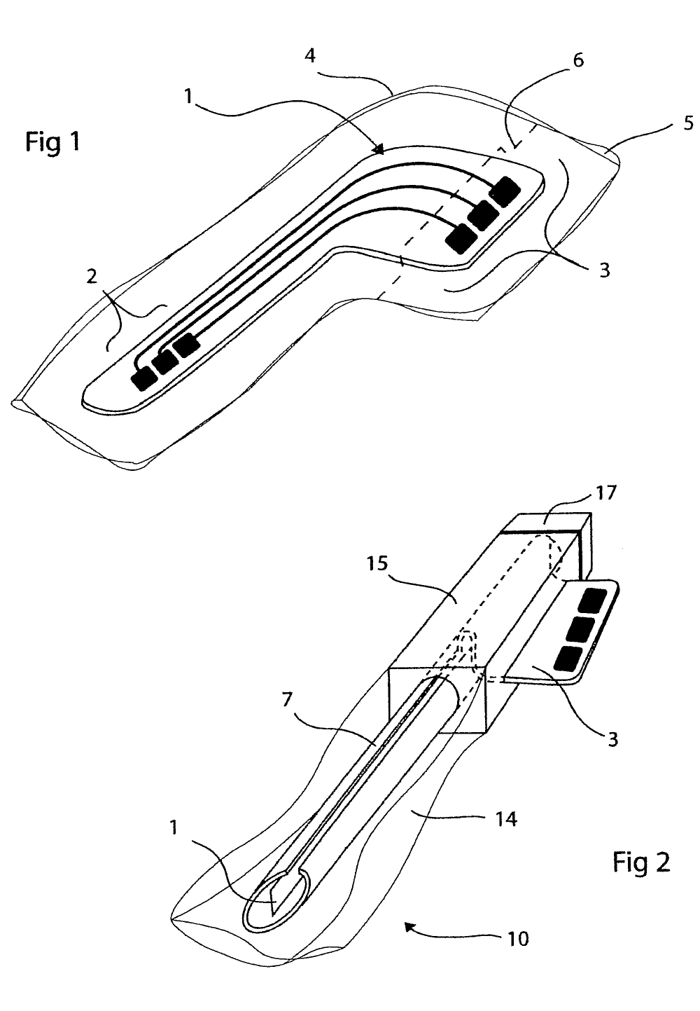

FIG. 1 shows a first embodiment of the invention;

FIG. 2 shows a second embodiment of the invention;

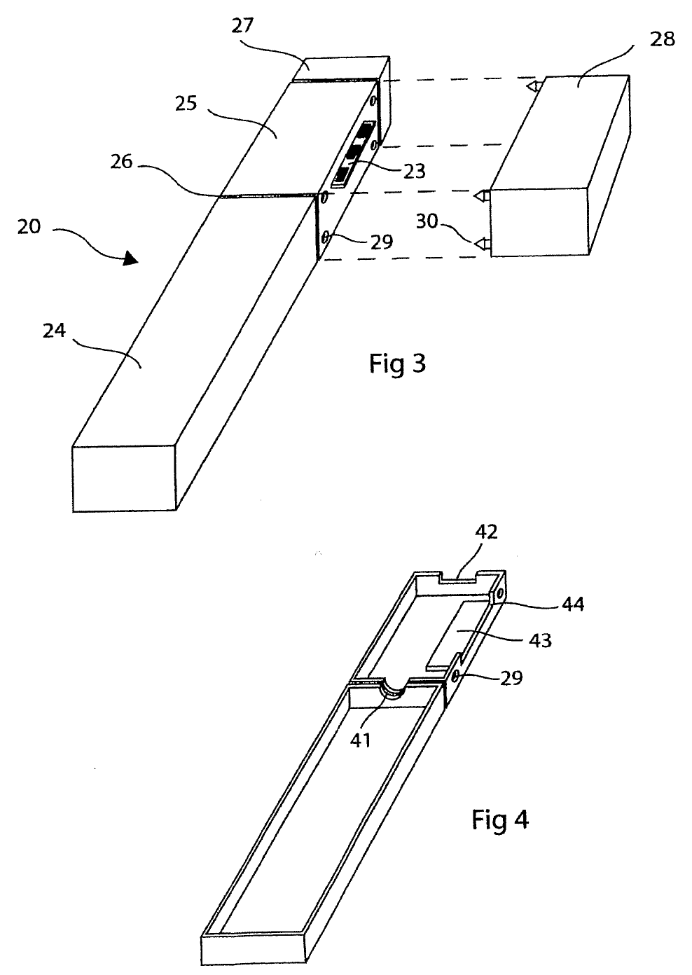

FIGS. 3 and 4 show details of an embodiment of the disposable sensor assembly according to the invention;

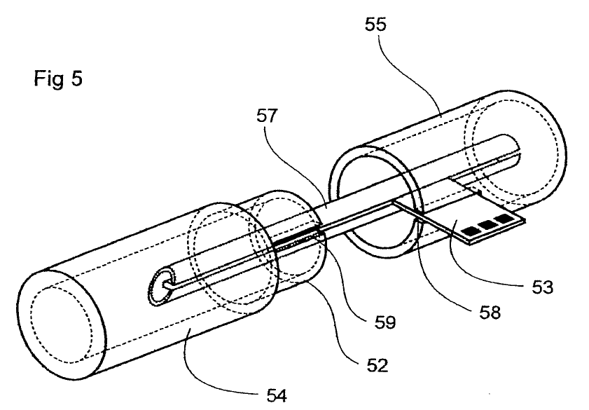

FIGS. 5-11 show further embodiments of the invention.

FIG. 1 shows a first embodiment of the sensor according to the invention. The figure shows a sensor 1 comprising an electrode area 2 and electric coupling area 3. Preferably the sensor is of the flexible type, wherein both electrode areas and electric coupling areas and the connections that connect areas electrically to each other are provided in a manner known per se by various kinds of printing or application techniques. The sensor 1 is sterile when supplied and is therefore shielded from the surroundings by means of a housing or a bag of a material which is sealed hermetically at least against micro-organisms. In FIG. 1, two shielded bags are shown, viz a so-called base packaging 4 and a supplementary packaging 5. The base packaging 4 is closely connected to the sensor 1 along the broken line 6, both on its top face and on its bottom face, thus readily enabling coupling of an electronic unit to the electric coupling areas 3, while simultaneously the remainder of the sensor—in particular the part that is to be implanted in the body—continues to be sterile. It will be understood that the line 6 can be situated in many other places as long as the sensor electrodes are separated from the electric coupling areas. However, it may be advantageous if the electric coupling areas 3 themselves are shielded to be in sterile condition by means of the further packaging 5 when the sensor is supplied to a user, since it is possible to tear off the further packaging 5 along a tearing zone, eg the one shown by broken line 6.

The invention solves two problems at a time, the first of which relates to the fact that the users are, on the one hand, private individuals, and, on the other, production lines for production of sterile sensor assemblies comprising sensor and electronics.

The second problem addressed by the invention is that of the sterilisation process being very difficult and cost-intensive, due to such sensor, eg for measuring the blood glucose level in a patient, comprising glucose oxidase on one of the electrodes and that, as well as other biological substances, are destroyed by the methods preferably used for sterilisation of electronics. For instance, ethylene oxide (EtO) will ruin the sensor, but will be suitable for sterilisation of electric circuits. In return, the electric circuits will be ruined eg by eg electron bombardment which is the preferred method for sterilising electrodes comprising microbiological substances; a method which is also very costly.

These problems are solved by the invention and in such a manner as to enable inexpensive and reliable manufacture of disposable sensors with disposable electronics coupled thereto.

Not before long, the electronic components used will become so inexpensive that it is financially viable to manufacture sensors with integrated electronics for single use. At present disposable sensors are most widely used that have to be interconnected with electronic circuits that are to be used multiple times. This introduces a need for manipulating the sensor in relation to the electric coupling while simultaneously, as mentioned, the part to be implanted in the body has to remain sterile. The advantages of the invention rely on the fact that the sensor area is sterilised and separated from the electric coupling area of the sensor by means of a shielding packaging or housing that protects the electrodes of the sensor by being destroyed when the electronics are sterilised or when the user is to mount his multiple-use electronics.

FIG. 2 shows an embodiment wherein the sensor also comprises a needle 7 for introducing the electrode area 2 into the body. It is a well-known technique to fold the flexible sensor 1 within a slit, hollow needle, whereby the sensor is implanted in the body simultaneously with the needle. The needle can subsequently be withdrawn, while simultaneously the coupling area 3 of the sensor can be arranged along the slit part of the needle. According to the invention, at least those parts of the sensor assembly 10 that are to be implanted in the body are sterile and shielded by means of a base packaging 14 that may be a flexible film attached to a housing 15 which is made of a form-stable material. According to the invention, the electric coupling areas project outside the housing 15, meaning that they need not be in a sterile condition when supplied, but this could be remedied by packing of the entire sensor assembly 10 in a sterile bag in the same manner as was suggested in connection with FIG. 1.

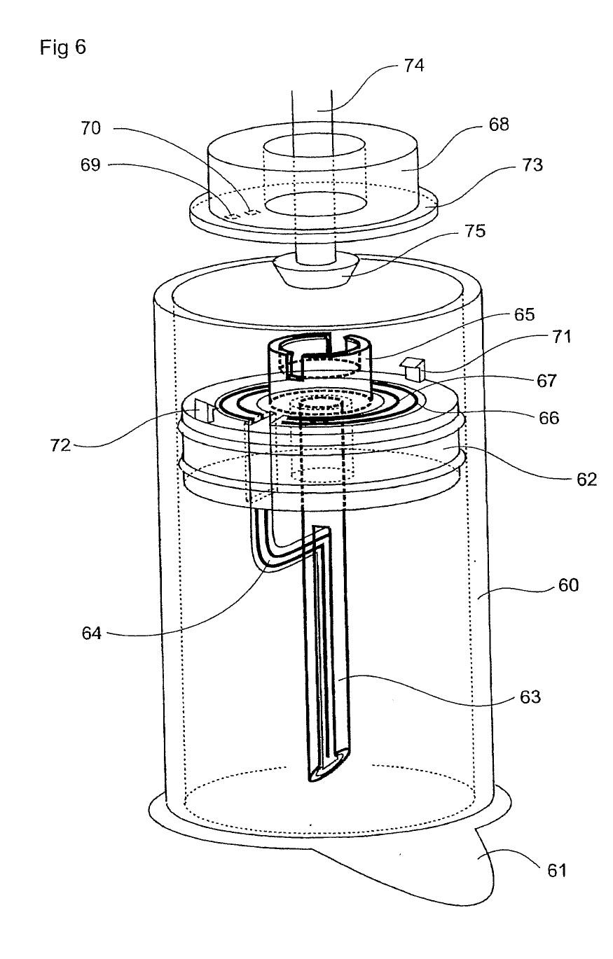

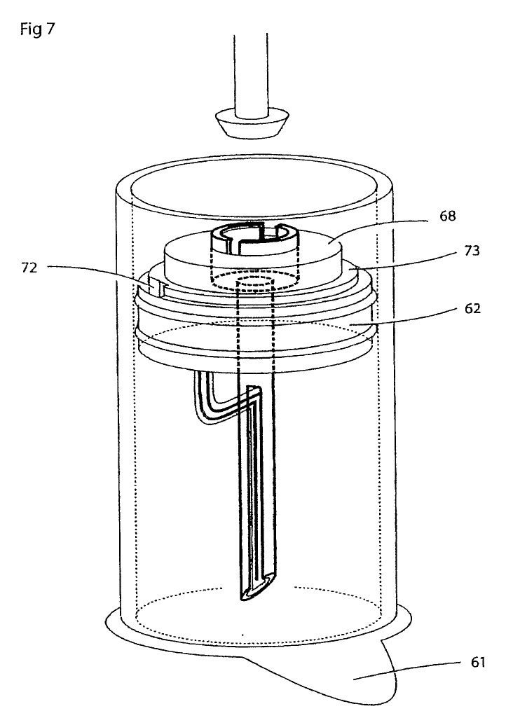

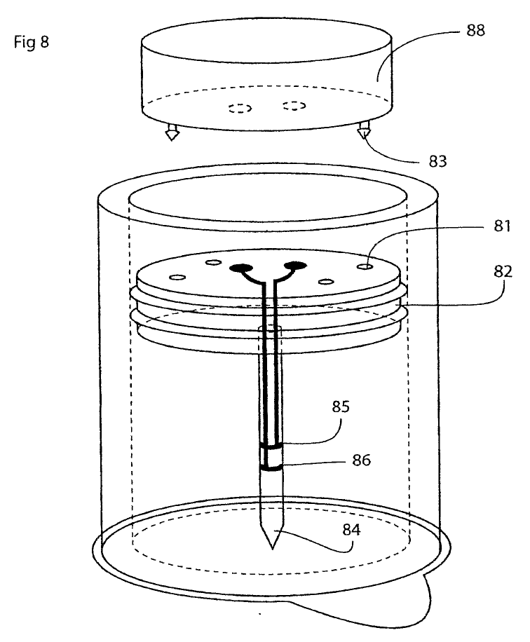

FIG. 2 shows an end portion 17 secured to the needle 7 and in case the housing 15 is supplied in sterile condition, it is an option to arrange a tape around the rim between the housing 15 and the end portion 17. If the housing 15 need not be sterile it is necessary to make sure that bacteria are unable to spread along the slit in the needle 7, see the explanation to FIGS. 6 and 7.

The embodiment shown in FIG. 2 is preferably suitable for use by private individuals that use disposable sensors in connection with multiple-use electronics. The multiple-use electronics will therefore not be sterile when the coupling takes place, and thus the invention provides a convenient simplification in that a part of the sensor is supplied in sterile condition and that the electric coupling areas of the sensor need not necessarily be aseptic, at least when they are to be interconnected with the electronics.

The advantages of the embodiment of FIG. 1 are most prominent in that it enables supply of an inexpensive sensor also for use by hospitals and doctors, where the sensor can be handled reliably in sterile condition in connection with implantation needles and optionally sterile electronics. Hereby the invention makes it possible to use fully implantable sensors with electronics coupled thereto, eg pacemakers. Thus, it is possible to connect a sterile sensor to an electronic circuit and then to sterilise the assembly in a hospital, ie outside a production line for sensor assemblies.

FIGS. 3 and 4 show embodiments of the invention that are particularly suitable for the supply of disposable sensors with electronics coupled thereon. According to the invention, an inexpensive and reliably sterile production process is accomplished.

FIG. 3 shows a sterile packaging comprising a lower part 24, a top part 25, and an end piece 27, which parts are comparable to parts 14, 15 and 17 of FIG. 2. According to the invention, parts 24 and 25 are manufactured to be coherent and of a form-stable material with an annularly extending rupture zone 26, and wherein the end piece 27 can be connected in a sterile manner to the housing 25 by means of a section of annularly extending tape. According to the invention the electric coupling area projects outside the part 25 as shown by 23, and FIG. 3 also shows a housing 28 accommodating electronic circuits to be connected to the electric coupling areas 23. Some time during the production process, the sterile sensor 20 has to be interconnected with the electronics in the housing 28 to form a permanent disposable sensor with electronics that can subsequently be sterilised—eg by means of ethylene oxide—due to the housing 24, 25 protecting the sensor against the destructive impact of the gas.

FIG. 4 shows one half-part of a packaging of the kind described in FIG. 3. The packaging consists of two laterally reversed half-parts for receiving sensor and needle, following which the half-parts are joined by welding and the entire sensor assembly is sterilised. By 41 is designated a bed (half of the bed) for a needle, and 42 designates half a recess for receiving a guide protrusion in the end piece, eg the one shown for 27 in FIG. 3. FIG. 4 also shows the coupling means 29 referred to above, and finally a support 43 is shown for supporting the sensor film that carries the electric coupling areas that are thus caused to be situated within the housing 25 which is also provided with a recess 44 for receiving projecting coupling means on the housing 28 in FIG. 3.

Preferably it will be arranged such that, when arranged on the support 43, the electric coupling area 23 is separated from the housing 24 in such a manner that, following the interconnection procedure, the sensor assembly can be sterilised, eg by means of ethylene gas, without any gas being allowed to come into contact with the sensor electrodes.

FIG. 5 illustrates a preferred embodiment which is intended in particular for a user himself to couple his multiple-use electronics to the electric coupling area 53. The needle or the insertion means 57 for the sensor is secured to a top part 55 having a slit 58 for receiving the electric coupling area 53. A lower portion 54 is configured for receiving the needle 57 and has a collar 52 of reduced diameter that can be received within the top part 55. The cooperating faces fit with each other, whereby a connection is formed in the interconnecting procedure that is impermeable to micro-organisms. The collar 52 is also provided with a slit 59 which is just as long as and flush with the slit 58, said slits adjoins the electric coupling area 53 in such a manner that it does not allow passage of micro-organisms, and thereby results in a double-sealing against micro-organisms.

The embodiment shown in FIG. 5 is particularly user-friendly since, preferably, the first step is to couple the multiple-use electronics to the electric coupling area 53 following which the sterile packaging is to be ruptured as parts 54 and 55 are pulled away from each other. Then the top part 55 can be used to hold on to when the needle is inserted into the body, and if one subsequently holds on to the electric coupling area 53 or the electronic equipment associated there with, the top part 55 can be withdrawn to the effect that the insertion part 57 is withdrawn from the body while simultaneously the electric coupling area leaves the top part 55 via the slit 58. Finally the top part 55 and the lower part 54 can be coupled to each other, thus protecting the used needle prior to it being discarded.

FIGS. 6 and 7 show an embodiment which is convenient both for serial production of the sensor assembly and for domestic use in connection with multiple-use electronics.

By this embodiment the shielding consists of a tube 60 being at the bottom closed by means of a tear-off label 61 and at the top being able to receive a piston 62 provided with O-rings, whereby it ensures sealing in a micro-organism-impermeable manner to the interior side of the tube 60, while simultaneously the piston 62 is displaceable within the tube 60. The piston 62 constitutes a part of the sensor which is, in FIGS. 6 and 7, of the flexible type, ie wherein a slit needle 63 or some other means of insertion is provided for introducing the sensor 64. The needle is slit only for some length for receiving the electrode area of the sensor and is otherwise solid or the like to pass by the piston 62 in a sealing manner that does not allow passage of micro-organisms; and on the other side of the piston the needle 63 is secured to an activator crown. By this embodiment it is very easy to ensure micro-organism-impermeable closure around the needle, since—compared to the embodiments described earlier—the sensor is conveyed around the needle and through piston, where the electric contact areas are situated in the form of conductive, concentric rings 66, 67.

The reference numeral 68 designates a housing for an electronic circuit and having has contact faces 69, 70, whose radial distances match those of the conductive rings 66, 67, whereby the electronic circuit 68 can be arranged in any rotational position in relation to the piston 62. The piston is provided with coupling means 71, 72 for cooperating with a bead 73 on the housing, and centrally both parts feature a cut-out for receiving an activator rod 74 with coupling head 75.

When the sensor shown in FIG. 6 is used in connection with multiple-use electronics 68, the circuit 68 may first be conveyed down into the tube 60, and by coupling of the head 75 to the crown 65 it is possible to hold back the piston, while the circuit 68 is pressed down to the effect that the bead 73 cooperates with the coupling means 71 and 72. Then the label 61 is pulled off and by means of the rod 74 the needle with sensor is pressed into the body. By holding on to the rod 74, the tube 60 can be withdrawn and finally the needle 63 can be withdrawn by means of the rod 74.

In case of a sensor assembly being produced in a production line, reference is made to FIG. 7, where the sterilisation process is explained.

Starting point now being taken in the same semi-finished product as was explained in the context of FIG. 6, viz the tube 60 with piston 62 and sensor and needle 63, 64 and label 61, sterilisation of the parts is performed within the tube 60 by radiation. Then, in the production line and by means of automated machinery performing the same actions as were explained above, it is possible to mount the electric circuit 68 to occupy the position shown in FIG. 7, following which the entire assembly is sterilised by means of ethylene oxide. According to the invention those parts of the sensor that do not tolerate exposure to ethylene oxide are protected within the cavity of the cylinder between label 61 and piston 62. Finally it is possible to apply a further shielding packaging around the sensor assembly as explained in the context of FIG. 1.

It is well-known that glucose oxidase and other substances used on electrodes are influenced by the sterilisation radiation in a manner which is difficult to predict. Thus, in accordance with the invention it is convenient, once a series of sensors has been made, to extract test samples and perform a measurement on the extracted sensors to determine an average correction factor that is to take into account the changes in the glucose oxidase. Such changes will be very similar for the entire series, and therefore information relating to correction parameters is particularly advantageously incorporated in all of the sensors in the series for the electronic circuits to use. This can be accomplished in a very simple manner, eg by an electronically readable label being applied onto the top face of the piston 62 as such information can be detected by the electronic circuit 68—either wirelessly or via a galvanized connection.

The wireless registration may be accomplished eg in that the label contains a pattern of conductors that constitutes an antenna area being able to reflect information contents back to the transmitter in the electronic circuit 68. Other options that are feasible in connection with a production line is that the electronic circuit 68 is coded with the correction parameters that apply to the sensors that are precisely to be provided with circuits 68 and sterilised. Preferably the circuit 68 is configured to transmit information on to a portable receiver.

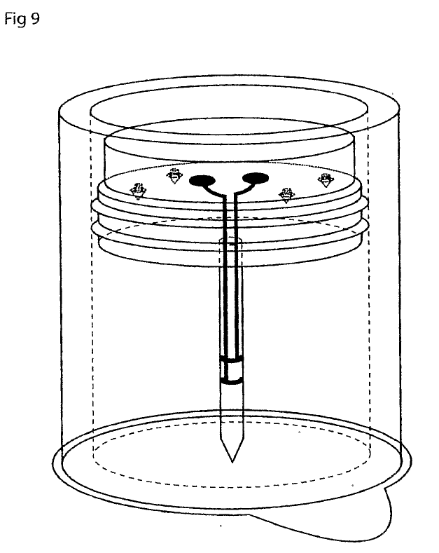

FIGS. 8 and 9 show a very simple embodiment as it contains a needle 84 configured as a sensor with electrodes 85, 86. The piston 82 therefore becomes very simple to manufacture and it can be coupled to a housing for the electronic circuit 88, eg by coupling means 81 and 83, see FIG. 9. The embodiment shown in FIG. 8 is—like the one shown in FIGS. 6 and 7—very suitable both as semi-manufactured product in a production line and as an inexpensive, user-friendly sensor.

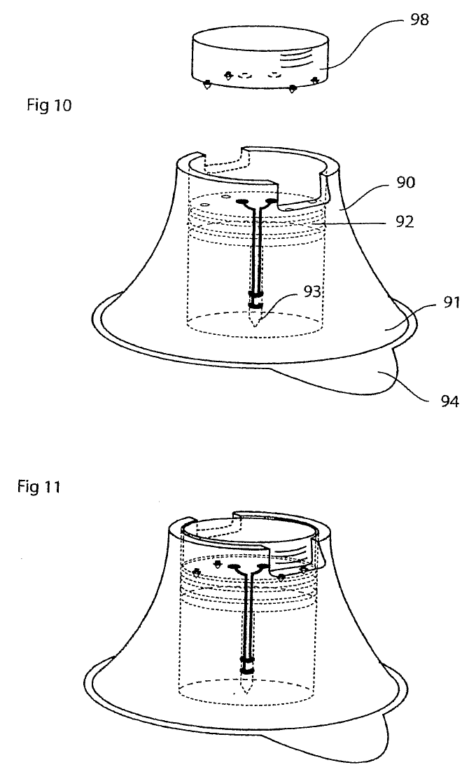

The embodiment shown in FIGS. 10 and 11 are first and foremost advantageous in the manufacture of a disposable sensor assembly. In the drawing the vertical dimensions are exaggerated for clarity, the sensor being in reality more flat. The previously described tube 60 or 80 constitutes in itself a conical part 90 of a sensor for being arranged in the body. The lowermost part of the body 90 has a base 91 with a plane lower side which is provided with an adhesive, whereby the sensor can be adhered to the body following removal of a label 94 corresponding to label 61 to expose the adhesive. The reference numeral 98 designates a housing for electronics that can be pressed down into the tube 90 and coupled to the piston 92, see FIG. 11, and thus corresponds to the parts shown by 68 and 62 in FIG. 7. By using a needle 93 of the same kind as was shown in the context of FIG. 8, the sensor assembly shown in FIGS. 10 and 11 can be made to be very simple and inexpensive to sterilise in accordance with the principles underlying the invention.

1. A method of forming a sterilised sensor package for an electrochemical sensor, said method comprising the steps of:
providing a sterilised sensor having an electrode area and an electric contact area,
forming a shielding packaging that is impermeable to micro-organisms in such a manner that at least the electrode area is enclosed in the shielding packaging, the electric contact area extending outside the shielding packaging.

2. A method according to claim 1, wherein at least the electrode are of the sensor is sterilised by radiation.

3. A method according to claim 1, wherein the shielding packaging is impermeable to gas.

4. A method according to claim 1, and wherein the sensor comprises electronics, and wherein the method further comprising the step of connecting the electronics to the electric contact area succeeding sterilisation of the electrode area of the sensor.

5. A method according to claim 1, wherein the electronics are sterilised and enclosed by a further shielding packaging.

6. A method according to claim 3, wherein the electronics are sterilised by means of a gas.

7. A method according to claim 6, wherein the gas is ethylene oxide (EtO).

8. A method of sterilising a series of sensors according to claim 1,
wherein the test samples of the sterilised sensors are extracted; that calculation of calibration parameters is performed by measurement; and that the part of the sensors that projects outside the shielding packaging is provided with an information carrier that contains the calibration parameters.

9. A method according to claim 8, wherein passive circuits are used as information carrier.

10. A method according to claim 8, wherein active electronic circuits are used as information carrier.

11. A sensor package comprising an implantable sensor having electrodes and electric coupling areas, which sensor is kept temporarily sterile by means of a shielding packaging or housing, wherein a base packaging is provided that shields at least the electrodes; and that the electric coupling areas are situated outside the base packaging.

12. A sensor package according to claim 11, wherein an additional packaging is provided configured for completely or partially shielding those parts of the sensor that project outside the base packaging.

13. A sensor package according to claim 12, wherein the additional packaging encloses the base packaging.

14. A sensor package according to claim 11, wherein the base packaging contains means for transdermal insertion of the electrodes of the sensor.

15. A sensor package according to claim 12, wherein the base packaging and the additional packaging adjoin each other along an endless rim comprising a tearing line or a rupture zone.

16. A sensor package according to claim 12, wherein at least a part of the base packaging is constituted by a form-stable housing.

17. A sensor package according to claim 14, wherein the means for transdermal insertion is a needle which is supported by the form-stable housing in such a manner as to enable withdrawal of the needle.

18. A sensor package according to claim 11, wherein the base packaging constitutes a form-stable sensor housing; and that a rupture zone is provided in the housing between the electrode part and the electric coupling part of the sensor.

19. A sensor package according to claim 18, wherein the housing comprises a pair of polymeric half-parts configured for being joined and intended for receiving a sensor and means for implanting the sensor, wherein the part of the housing next to the electric coupling areas is configured for supporting the means for implanting the sensor in such manner that said means will be released during a withdrawal process.

20. A sensor package according to claim 19, wherein the part of the housing next to the electric coupling areas has mechanical coupling means for co-operating with a housing containing an electronic circuit.

21. A sensor package according to claim 11, wherein the base packaging constitutes a form-stable housing comprising and upper part and a lower part, which parts can be shifted a distance into each other by means of co-operating slide faces that constitute a barrier which is impermeable to micro-organisms when the parts are interconnected, wherein the upper part is secured to the means for transdermal insertion of the sensor and has a slit being open towards the lower part for slideably receiving the electric contact area of the sensor.

22. A sensor package according to claim 11, wherein the base packaging comprises a tube which is closed at the one end and the opposite end of which is configured for receiving a part of the sensor which is configured as a piston, which piston in combination with the interior wall of the tube constitutes a slide seal impermeable to micro-organisms and is situated between the electrodes and the electric coupling areas of the sensor.

23. A sensor package according to claim 22, wherein the tube is closed at the one end by means of a tear-off label.

24. A sensor package according to claim 22, wherein the piston is a support for a needle sensor.

25. A sensor package according to claim 24, wherein the piston face that faces away from the needle has electric coupling areas and has mechanical coupling means for cooperating with a housing for electronic circuits, whereby the electronic circuits can be interconnected with the electric coupling areas.

26. A sensor package according to claim 22, wherein the closed end of the piston has a base with an adhesive intended for adhesion of the tube to the body; and that the tear-off label is secured by that adhesive.

27. A sensor package according to claim 22, wherein the sensor is flexible; and that means are provided for transdermal insertion of the sensor, said means being slideably supported in the piston.

28. A sensor package according to claim 27, wherein the means for inserting the sensor is a needle having a longitudinal slit for receiving the electrode area of the sensor, said slit extending from the distal end of the needle, but not beyond the piston.

29. A sensor package according to claim 22, wherein the electric contact areas on the piston comprise concentric rings of electrically conductive material for cooperating with electric switches on a housing for an electronic circuit.

30. A sensor package according to claim 22, wherein the side of the piston that faces away from the sensor has mechanical coupling means for cooperating with a housing for an electronic circuit and means for cooperating with a rod for insertion of the sensor.
